(19) United States
(12) Patent Application Publication
(10) Pub. No.: US 2009/0298747 A1
(43) Pub. Date: Dec. 3, 2009

(54) Methods and Compositions For Treatment of Nitric Oxide-Induced Clinical Conditions

(75) Inventor: Leland Shapiro, Denver, CO (US)

Correspondence Address: Don D. Cha, 547 Buena Vista Road, Golden, CO, 80401, US

(73) Assignee: Bio Holding, Inc., Wilson, NC (US)

(21) Appl. No.: 12/427,075

(22) Filed: Apr. 21, 2009

Related U.S. Application Data
(62) Division of application No. 09/518,076, filed on Mar. 3, 2000.
(60) Provisional application No. 60/123,167, filed on Mar. 5, 1999.
(60) Provisional application No. 60/153,942, filed on Sep. 15, 1999.

(51) Int. Cl. A61K 38/14; A61P 29/00; A61P 11/00; A61P 9/00

(52) U.S. Cl. 514/8

(57) ABSTRACT

The present invention provides compositions and methods for modulating cellular nitric oxide (NO) production and for treating a clinical condition associated therewith.

### CROSS-REFERENCE TO RELATED APPLICATIONS

This is a continuation-in-part application of U.S. patent application Ser. No. 09/518,076, filed Mar. 3, 2000, which claims the priority benefit of U.S. Provisional Application Nos. 60/123,167, filed Mar. 5, 1999, and 60/153,942, filed Sep. 3, 1999, all of which are incorporated herein by reference in their entirety.

### FIELD OF THE INVENTION

The present invention relates to compositions and methods for modulating cellular nitric oxide (NO) production and for treating a clinical condition associated therewith.

### BACKGROUND OF THE INVENTION

Serine proteases serve an important role in human physiology by mediating the activation of vital functions. In addition to their normal physiological function, serine proteases have been implicated in a number of pathological conditions in humans. Serine proteases are characterized by a catalytic triad consisting of aspartic acid, histidine and serine at the active site.

The naturally occurring serine protease inhibitors are usually, but not always, polypeptides and proteins which have been classified into families primarily on the basis of the disulfide bonding pattern and the sequence homology of the reactive site. Serine protease inhibitors, including the group known as serpins, have been found in microbes, in the tissues and fluids of plants, animals, insects and other organisms. Protease inhibitor activities were first discovered in human plasma by Fermi and Pemossi in 1894. At least nine separate, well-characterized proteins are now identified, which share the ability to inhibit the activity of various proteases. Several of the inhibitors have been grouped together, namely α₁-proteinase inhibitor, antithrombin III, antichymotrypsin, C1-inhibitor, and α₂-antiplasmin, which are directed against various serine proteases, i.e., leukocyte elastase, thrombin, cathepsin G, chymotrypsin, plasminogen activators, and plasmin. These inhibitors are members of the α₁-proteinase inhibitor class. The protein α₂-macroglobulin inhibits members of all four catalytic classes: serine, cysteine, aspartic, and metalloproteases. However, other types of protease inhibitors are class specific. For example, the α₁-proteinase inhibitor (also known as α₁-antitrypsin or α1-antitrypsin) and inter-α-trypsin inhibitor inhibit only serine proteases, α₁-cysteine protease inhibitor inhibits cysteine proteases, and α₁-anticollagenase inhibits collagenolytic enzymes of the metalloenzyme class.

Human neutrophil elastase (NE) is a proteolytic enzyme secreted by polymorphonuclear leukocytes in response to a variety of inflammatory stimuli. The degradative capacity of NE, under normal circumstances, is modulated by relatively high plasma concentrations of α₁-antitrypsin. However, stimulated neutrophils produce a burst of active oxygen metabolites, some of which (hypochlorous acid for example) are capable of oxidizing a critical methionine residue in α₁-antitrypsin. Oxidized α₁-antitrypsin has been shown to have a limited potency as a NE inhibitor and it has been proposed that alteration of this protease/antiprotease balance permits NE to perform its degradative functions in localized and controlled environments.

α₁-Antitrypsin is a glycoprotein of MW 51,000 with 417 amino acids and 3 oligosaccharide side chains. Human α₁-antitrypsin was named anti-trypsin because of its initially discovered ability to inactivate pancreatic trypsin. Human α₁-antitrypsin is a single polypeptide chain with no internal disulfide bonds and only a single cysteine residue normally intermolecularly disulfide-linked to either cysteine or glutathione. The reactive site of α₁-antitrypsin contains a methionine residue, which is labile to oxidation upon exposure to tobacco smoke or other oxidizing pollutants. Such oxidation reduces the biological activity of α₁-antitrypsin; therefore, substitution of another amino acid at that position, e.g., alanine, valine, glycine, phenylalanine, arginine or lysine, produces a form of α₁-antitrypsin which is more stable. Some of the important amino acids near the carboxy terminal end of α₁-antitrypsin are those at positions 393-397.

The C-terminus of human α₁-antitrypsin is homologous to antithrombin (ATIII), antichymotrypsin (ACT), C1-inhibitor, tPA-inhibitor, mouse anti-trypsin, mouse contrapsin, barley protein Z, and ovalbumin. The normal plasma concentration of ATT ranges from 1.3 to 3.5 mg/mL although it can behave as an acute phase reactant and increases 3-4-fold during host response to inflammation and/or tissue injury such as with pregnancy, acute infection, and tumors. It easily diffuses into tissue spaces and forms a 1:1 complex with a target protease, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen. Humans with circulating levels of α₁-antitrypsin less than 15% of normal are susceptible to the development of lung disease, e.g., familial emphysema, at an early age. Familial emphysema is associated with low ratios of α₁-antitrypsin to serine proteases, particularly elastase. Therefore, it appears that this inhibitor represents an important part of the defense mechanism against attack by serine proteases.

α₁-Antitrypsin is one of few naturally occurring mammalian serine protease inhibitors currently approved for the clinical therapy of protease imbalance. Therapeutic α₁-antitrypsin has been commercially available since the mid 80's and is prepared by various purification methods (see, for example, U.S. Pat. Nos. 4,629,567; 4,760,130; 5,616,693; and PCT Publication Number WO 98/56821). Prolastin is a trademark for a purified variant of α₁-antitrypsin and is currently sold by Talecris Company. Recombinant unmodified and mutant variants of α₁-antitrypsin produced by genetic engineering methods are also known (see, for example, U.S. Pat. No. 4,711,848); methods of use are also known, e.g., α₁-antitrypsin gene therapy/delivery (see, for example, U.S. Pat. No. 5,399,346).

The two known cellular mechanisms of action of serine proteases are by direct degradative effects and by activation of G-protein-coupled proteinase-activated receptors (PARs). The PAR is activated by the binding of the protease followed by hydrolysis of specific peptide bonds, with the result that the new N-terminal sequences stimulate the receptor. The consequences of PAR activation depend on the PAR type that is stimulated and on the cell or tissue affected and may include activation of phospholipase α₁-activation of protein kinase C and inhibition of adenylate kinase.

Nitric oxide (NO), also known as endothelium-derived relaxing factor (EDRF), is a potent vasodilator, oxidant, and neurotransmitter produced by many different types of cells and tissues, such as endothelium, macrophages and neuronal cells. Based on DNA analyses, it is believed that the NO synthase enzymes (NOS) exist in at least three isoforms, namely, neuronal constitutive NOS (N-cNOS) which is present constitutively in neurons, endothelial constitutive NOS (E-cNOS) which is present constitutively in endothelial cells, and inducible NOS (iNOS) which is expressed following stimulation by cytokines and lipopolysaccharides in macrophages and many other cells. Among these three isoforms, N-cNOS and E-cNOS are calcium-dependent whereas iNOS is calcium-independent. NO synthesized by nitric oxide synthase from arginine and oxygen is also an important signal transducing molecule in various cell types. In macrophages NO has assumed, under certain situations, the role of a cytotoxic agent-a reactive nitrogen intermediate that is lethal to cancer cells and microorganisms.

The release of nitric oxide is also involved in other acute and chronic inflammatory diseases. These diseases include but are not limited to diseases such as, for example, acute and chronic infections (viral, bacterial and fungal), acute and chronic bronchitis, sinusitis, and upper respiratory infections, including the common cold; acute and chronic gastroenteritis and colitis; acute and chronic cystitis, and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystitis; acute and chronic vaginitis; drug reactions; insect bites; burns and sunburn.

Released NO combines very rapidly with superoxide to form peroxynitrite (ONOO⁻•), a reactive tissue damaging nitrogen species thought to be involved in the pathology of several chronic diseases. Peroxynitrite nitrates tyrosine residues and inactivates α₁-antitrypsin. This mechanism is postulated to be responsible for α₁-antitrypsin inactivation by cigarette smoke. Nitric oxide inhibits iron-containing enzymes important in respiration and DNA synthesis. Peroxynitrite decomposes to the reactive NO₂ and hydroxyl radicals, and NO stimulates ADP-ribosylation of various proteins including glyceraldehyde-3-phosphate dehydrogenase, with consequent inactivation.

It has been shown that the acute phase protein α₁-antitrypsin inhibits the cellular lethality induced by tumor necrosis factor (TNF) both in normal mice and in mice sensitized with galactosamine but similar apoptosis of hepatocytes induced by anti-Fas remained unaffected. However, α₁-antitrypsin did not affect the induction by TNF of NO. In contrast, others have shown that TNF injury was not prevented by α₁-antitrypsin.

Many proteins are reported to modulate NO production. Macrophage deactivating factor and TGF-β partially blocked NO release by macrophages activated with γ-interferon (γ-IFN or IFN-γ) and TGF-α (transforming growth factor-α), but not when activated by γ-IFN and lipopolysaccharide (LPS or endotoxin). Epidermal growth factor can suppress both NO and H₂O₂ production by keratinocytes. Incubation of LPS-activated peritoneal neutrophils with IL-8 blocks both the release of NO and NOS induction at the transcriptional level.

TGF-β₁ and 12-O-tetradecanoylphorbol-13-acetate (i.e., phorbol myristyl acetate or PMA) inhibit LPS and γ-IFN-induced NO synthesis in mouse bone marrow cells. In contrast, in bovine pigmented retinal epithelial cells TGF-β₁ increases the NO production, as measured by nitrite, attributable to treatment with LPS and 7-IFN. In this system both fibroblast growth factor (FGF-1 and FGF-2) inhibit nitrite production, likely by inhibiting the induction of NOS mRNA at the transcriptional level. Insulin-like growth factor 1 reduces the amount of NO produced by the action of IL-1_{β} on vascular smooth muscle cells. The fact that so many agents can modulate NO activity by increasing or inhibiting NO production suggests that NO production may be important in many different contexts.

The overproduction in the body of nitric oxide (NO) and/or peroxynitrite (ONOO—^{•}) has been suggested by some to be a contributing factor to diseases that are immune-mediated and/or inflammatory. In a clinical study, the levels of IL-6, IL-1_{β}, NO and α₁-antitrypsin were shown to be involved in the pathogenesis of scorpion envenomation and correlated with the severity of envenomation. An extensively used model system to study multiple sclerosis, an example of a disease treated by the present invention, is experimental allergic encephalomyelitis (EAE) in rats and mice.

Thus, the prior art taught that NO metabolites inactivate α₁-antitrypsin. Also taught was that in certain clinical situations NO levels tended to rise concomitantly along with increase in α₁-antitrypsin levels, although the α₁-antitrypsin activity may have been reduced. However, the prior art failed to recognize that α₁-antitrypsin might in fact prevent NO synthesis. The present inventor discovered that therapeutic and physiological levels of α₁-antitrypsin can efficiently block γ-IFN- and LPS-induced NO synthesis. This invention addresses a long-felt need for safe and effective amelioration of many diseases related to nitric oxide-caused damage.

### BRIEF DESCRIPTION OF THE DRAWINGS

FIG. 1 illustrates the effect of α₁-antitrypsin on NO release upon induction with LPS and γ-IFN.

FIG. 2 illustrates the effect of α₁-antitrypsin on induction of iNOS protein by LPS and γ-interferon.

FIG. 3 illustrates an electrophoretic mobility shift assay of NF-κB on gel electrophoresis demonstrating inhibition of NF-κB activation due to the presence of α₁-antitrypsin.

FIG. 4 illustrates the inhibition of elevated NO levels, measured as NO₂⁻, by CE-2072.

FIG. 5 illustrates the inhibition of p-ERK expression by α₁-antitrypsin (α1-antitrypsin).

### DETAILED DESCRIPTION OF THE INVENTION

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2d ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Various biochemical, molecular biology, microbiology, and recombinant DNA techniques methods are well known in the art. For example, methods of isolation and purification of nucleic acids as well as recombinant DNA techniques are described in detail in WO 97/10365, WO 97/27317, Chapter 3 of Laboratory Techniques in Biochemistry and Molecular Biology Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); Current Protocols in Molecular Biology, (Ausubel, F. M. et al., eds.) John Wiley & Sons, Inc., New York (1987-1999), including supplements such as supplement 46 (April 1999); and Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

The terms “nucleic acid” “polynucleotide” and “oligonucleotide” are used interchangable herein and refer to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). A “subsequence” or “segment” refers to a sequence of nucleotides that comprise a part of a longer sequence of nucleotides.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

When referring to the context of two peptides, the terms “substantially identical” and “conserved” are used interchangeably herein and refer to two or more sequences or subsequences that have at least 80%, typically at least 90% or 95%, often at least 98%, 99% or higher peptide identity, when compared and aligned for maximum correspondence, as measured using a sequence comparison algorithm such as those described below for example, or by visual inspection. Typically, the substantial identity exists over a region of the active site sequences, and in yet other instances the sequences are substantially identical over the full length of the sequences being compared.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection [see generally, Current Protocols in Molecular Biology, (Ausubel, F. M. et al., eds.) John Wiley & Sons, Inc., New York (1987-1999, including supplements such as supplement 46 (April 1999)]. Use of these programs to conduct sequence comparisons are typically conducted using the default parameters specific for each program.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid is considered similar to a reference sequence if the smallest sum probability in a comparison of the test amino acid to the reference amino acid is less than about 0.1, typically less than about 0.01, and often less than about 0.001.

“Modulation” refers to a change in the level or magnitude of an activity or process. The change can be either an increase or a decrease. For example, modulation of gene expression includes both gene activation and gene repression. Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT, β-galactosidase, β-glucuronidase, green fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca²⁺), and cell growth.

“A derivative” of α₁-antitrypsin refers to a homolog, an analog of α₁-antitrypsin. Exemplary derivatives of α₁-antitrypsin include, but are not limited to: (i) peptides derived from α₁-antitrypsin; (ii) peptides in which one or several amino acids of the natural α₁-antitrypsin sequence have been substituted by other amino acids; (iii) α₁-antitrypsin modified at the N- and/or C-terminal end of the peptide sequence, for example, by substitution; thus, esters and amides can be considered as C-terminal derivatives; (iv) α₁-antitrypsin peptides the modification of which prevents their destruction by proteases or peptidases, as well as to peptide-PEG-conjugates derived from the basic sequence of α₁-antitrypsin or its fragment; (v) modified peptides which are derived from the chain of α₁-antitrypsin or its fragment and wherein one or several of the amino acids of the sequence have been substituted by genetically encoded or not genetically encoded amino acids or peptidomimetics. They may exist as free peptides or as C-terminal derivative and/or being linked to a polyethylene glycol (PEG)-polymer, or joined to an antibody component (either Fc or Fab) and have the desired α₁-antitrypsin effects; and (vi) peptides having conservative substitutions of amino acids as compared to the natural sequence of α₁-antitrypsin in one or several positions. A conservative substitution is defined as the side chain of the respective amino acid being replaced by a side chain of similar chemical structure and polarity, the side chain being derived from a genetically coded or not genetically coded amino acid. Families of amino acids of this kind having similar side chains are known in the art. They comprise for instance amino acids having basic side chains (lysins, arginins, histidine), acidic side chains (aspartic acid, glutamic acid), uncharged polar side chains (glycine, aspartamic acid, glutamine, serine, threonine, tyrosine, cysteine), non-polar side chains (alanine, valine, leucine, isoleucine, proline, phenylalanine, methionine, tryptophan), β-branched side chains (threonine, valine, isoleucine) and aromatic side chains (tyrosine, phenylalanine, tryptophane, histidine). Such conservative substitutions of side chains is typically carried out in non-essential positions. In this context, an essential position in the sequence is one wherein the side chain of the relevant amino acid is of significance for its biological effect.

Some aspects of the invention provide methods for treating a subject for a clinical condition associated with over expression of NO synthase, over production or elevated synthesis of nitric oxide. Unless the context requires otherwise, the terms “over expression of nitric oxide”, “over production of nitric oxide”, and “elevated synthesis of nitric oxide” are used interchangeably herein and refer the level of nitric oxide present in a subject that results in manifestation of a clinical condition such as a disease or a disorder. As discussed above, nitric oxide is naturally produced for various cellular activities including, but not limited, to cell signaling. In most instances, the production of nitric oxide does not result in any disease or disorder. However, an elevated level of NO results in manifestation of various clinical conditions as discussed above.

Inhibition of NO production has many important therapeutic benefits, as described infra. NO production contributes to septic shock, the adverse consequences of ischemia, inflammation including acne, hypotension, cell death and other physiological processes and effects. The cytokines IL-2 and TNF, which have significant potential as therapeutic agents to treat cancer, induce high levels of NO production, resulting in hypotensive shock. This adverse side effect is reversed by administering NO inhibitors with these cytokines. Thus, the functional agents of the invention may be useful as primary or ancillary therapeutic agents for the treatment of these and other NO-mediated diseases or disorders, or effects.

FIG. 1 illustrates a specific embodiment of the invention in which α₁-antitrypsin inhibits NO levels induced by the inflammatory mediators γ-interferon (γ-IFN) and lipopolysaccharide (LPS) in macrophagic cells. Analyses of inducible nitric oxide synthase expression reveal that the inflammatory mediators increase NO levels, and that α₁-antitrypsin inhibits the induction.

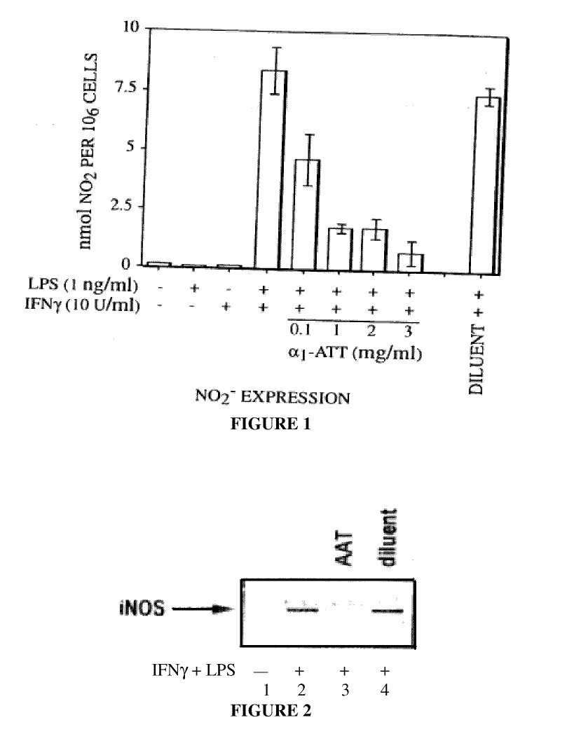

FIG. 2 illustrates another specific embodiment of the invention, in which α₁-antitrypsin inhibits induction of iNOS protein (one of the enzymes responsible for NO synthesis) induced by the inflammatory mediators γ-interferon (γ-IFN) and lipopolysaccharide (LPS) in macrophagic cells. Western blot analyses of inducible nitric oxide synthase expression reveals that the inflammatory mediators increase iNOS protein levels, and that α₁-antitrypsin inhibits the induction.

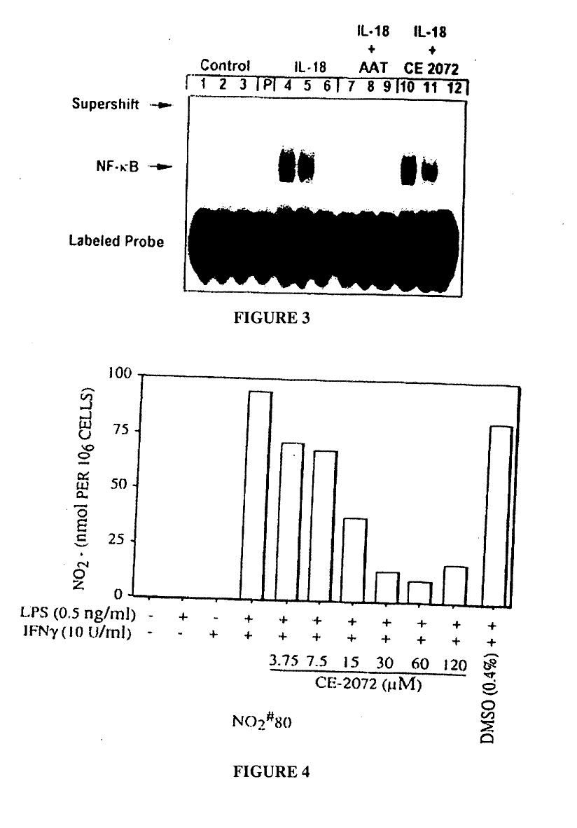

FIG. 3 shows the electrophoretic mobility shift due to nuclear factor-KB (NF-κB) induced by incubation with interleukin-18 (IL-18). NF-κB is a positive regulator of NOS induction. As shown in the figure, both α₁-antitrypsin and CE-2072 inhibit the induction of active NF-κB.

FIG. 4 illustrates yet another specific embodiment of the invention, in which CE-2072 inhibits NO levels resulting from induction of iNOS by IFN-γ and LPS. CE-2072, a peptoid with the structure benzyloxycarbonyl-L-valyl-N-[1-(2-[5-(3-methylbenzyl)-1,3,4-oxadiazolyl]-carbonyl)-2-(S)-methylpropyl]-L-prolinamide, is revealed in this figure to be an inhibitor of NO.

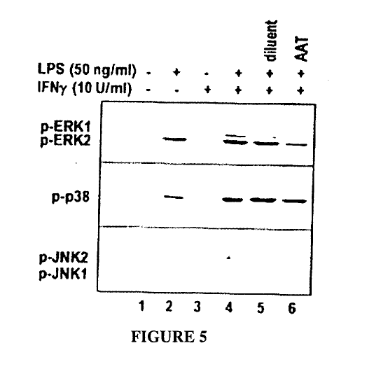

FIG. 5 is a Western blot showing α₁-antitrypsin inhibits the level and/or phosphorylation of p-ERK (phospho-extracellular signal regulated kinase, also termed p42/p44 MAP kinase. The figure is a Western blot (protein blot of SDS polyacrylamide electrophoresis) of p38 and p-ERK, and an autoradiograph of p-JNK SDS polyacrylamide electrophoresis.

Some aspects of the invention include administering two or more (e.g., two or three) independently acting agents. In some particular embodiments within these aspects of the invention, a composition comprising both (i) AAT or other serine protease inhibitor, and (ii) an antioxidant, a nitric oxide scavenger, or a peroxynitrite scavenger is administered.

Exemplary peroxynitrite scavengers include, but are not limited to, 2,6,8-trihydroxypurine (uric acid), dihydrorhodamine, and compounds that contain a thiol group (typically glutathione or cysteine). Uric acid is also considered to be a hydroxyl radical scavenger.

Anti-oxidants, including, but not limited to, vitamin A, vitamin E, vitamin C, cysteine, ω-3-unsaturated lipids, ω-6-unsaturated lipids, α-carotenes, β-carotenes, selenium, curcumin, a superoxide dismutase preparation, ginkgo biloba, lycopenes, glutathione, bioflavenoids, catechins, lignans, linolenic acid, quercetin, zeaxanthin, or combinations or complexes thereof, can also be used in conjunction with α₁-antitrypsin, a derivative thereof, or a combination thereof.

In yet another embodiment of the invention, superoxide-resistant α1-antitrypsin enzymes and forms of α1-antitrypsin are used to avoid inactivation by excess NO. As an example, synthetic α1-antitrypsin or recombinant α1-antitrypsin produced with alternative and oxidation-resistant amino acid sequences are embodiments of the invention.

NO can react and form ONOO⁻, which is known to inactivate α₁-antitrypsin. Therefore, any agent that replenishes α₁-antitrypsin activity through inhibition of NO production ameliorates diseases resulting from reduced α₁-antitrypsin activity. Accordingly, some aspects of the invention provide methods that use inhibitors of NO synthesis to indirectly protect the amount of active α₁-antitrypsin. Many inhibitors of NO are useful in this embodiment including derivatives of amino acids, for example, N^{G}-nitro-L-arginine methyl ester (L-NAME), N^{G}-nitro-L-arginine (L-NA), N^{G}-methyl-L-arginine (L-NA), N,N′-dimethylarginine, N^{G}-monoethyl-L-arginine acetate, N^{G}-monomethyl-L-arginine acetate, N^{G}-monomethyl-D-arginine, N^{G}-monomethyl-L-homoarginine acetate, N N^{G}-nitro-D-arginine, N^{G}-nitro-D-arginine methyl ester hydrochloride, N^{G}-nitro-L-arginine, and L-N⁶-(1-iminoethyl)lysine, and salts thereof.

In some embodiments, non-amino acid inhibitors of NO can be used in the compositions. Exemplary non-amino acid NO inhibitors include, but are not limited to, guanidine, guanidine derivatives, S-alkylisothioureas, amidines, imidazoles, indazoles, and mercaptoalkylguanidines, and salts thereof. Specific examples of non-amino acid NO inhibitors include aminoguanidine, S-methylisothiourea sulfate, S-ethylisothiourea sulfate, S-aminoethylisothiourea sulfate, mercaptoethylguanidine, 2,4-diamino-6-hydroxypyrimidine, diphenylene iodonium chloride, 2-ethyl-2-thiopseudourea hydrobromide, 2-iminobiotin, L-N⁵-(1-iminoethyl)ornithine hydrochloride, S-methyl-L-thiocitrulline dihydrochloride, p-nitroblue tetrazolium chloride, 3-bromo-7-nitroindazole, pentamidine isethionate, 1-pyrrolidinecarbodithioic acid, spermidine, spermine, spermine-NO, 3-morpholinosydonimine-N-ethylcarbamide, L-thiocitrulline, troleandomycin, and 7-nitroindazole, and salts thereof. However, it should be appreciated that the scope of the invention is not limited to these named examples. Furthermore, agents that bind NO are suitable for this embodiment of the invention and these agents can include, for example, heme-containing proteins including hemoglobin, myoglobin, cytochrome V, guanylyl cyclase, NADH:ubiquinone oxidoreductase, NADH:succinateoxidoreductase and cis-aconitase, and salts thereof. Certain agents that ordinarily function as donors of NO also have a paradoxical effect on the inhibition of NOS and are suitable for use in the sparing of α₁-antitrypsin. Suitable NO donor agents include S-nitroso-N-acetylpenicillamine, S-nitrosoglutathione and nitroglycerine.

### Diseases Addressed by the Invention

Specific diseases or disorders for which the therapeutic methods of the invention are beneficial include but are not limited to inflammatory diseases or disorders, hypotension, and the like. The disease or disorder can be selected from the group consisting of but not limited to acquired tubulointerstitial disease, acute pancreatitis, acute respiratory failure, acute respiratory distress syndrome (ARDS), age-associated memory impairment, AIDS, airway inflammation, Alzheimer's disease, amyotrophic lateral sclerosis, asthma, atherosclerosis, autoimmune disease, myocarditis, carcinogenesis, cerebral ischemia, cerebrovascular disease, chronic liver disease, chronic lung disease, chronic obstructive pulmonary disease, chronic otitis media, congestive heart failure, coronary artery disease, coronary artery ectasia, diabetes mellitus, diabetic neuropathy, dysfunctional uterine bleeding, dysmenorrhea, endotoxic shock, end-stage renal disease, falciparum malaria, gastric carcinogenesis, gastrointestinal pathophysiology, glaucoma, glutamate-induced asthma, glutamate induced Chinese restaurant syndrome, heart failure, heat stress, gastritis, hot-dog headache, Hirschsprung's disease, HIV infection, hypertension, hypoxemic respiratory failure, inflammatory arthritis, inflammatory bowel disease (Crohn's disease and ulcerative colitis), inflammatory joint diseases, liver cirrhosis, liver disease, Lyme neuroborreliosis, migraine, multiple sclerosis, neonatal and pediatric respiratory failure, nephrotoxicity, neurodegenerative diseases, orthopedic disease, osteoarthritis, oxidant stress, Parkinson's disease, pediatric pulmonary disease, pleural inflammation, preeclampsia, primary ciliary dyskinesia, primary pulmonary hypertension, protozoan infections, pugilistic Alzheimer's disease, pulmonary hypertension, retinal disease, septic shock, sickle cell anemia, rheumatoid arthritis, stroke, systemic lupus erythematosus, traumatic brain injury, tumor progression, or vascular disease. These diseases are thought to be mediated, at least in part, by aberrant levels of nitric oxide. In specific embodiments, the inflammatory disease or disorder is mediated at least in part by an agent selected from the group consisting of γ-interferon and lipopolysaccharide.

As noted above, the present invention can be used in the treatment of hypotension, including but not limited to hypotension resulting from septic, endotoxic, hypovolemic, or traumatic shock, chronic hypotension, and disorders associated with hypotension, such as priapism. Accordingly, the invention further provides for administering an amount of a vasoconstrictor NO antagonist effective to increase blood pressure in an animal in addition to or in conjunction with administration of α₁-antitrypsin, a derivative thereof, or a combination thereof. Suitable vasoconstrictors include, but are not limited to, epinephrine; norepinephrine; vasopressin; N^{G}-monomethyl-L-arginine (L-NMA); N^{G}-nitroarginine methylester (L-NAME), and thromboxane-A₂.

Additionally, a representative sample of diseases that the methods and compositions of the invention are to treat are listed in Table 1.

TABLE 1
Diseases Related to Excess NO

| NO Effect | Disease(s) |
|---|---|
| Decreased Blood pressure | Sepsis, septic shock, ARDS (shock lung), acute renal |
| (vasodilation) | failure, shock liver, acute ischemic bowel injury |
| Decreased cardiac output | Myocardial depression of sepsis, acute and chronic |
| | congestive heart failure |
| HIV production | HIV infection, AIDS |
| Production of ONOO⁻ | 1. Ischemic brain injury |
| (peroxynitrite) and reactive oxygen | 2. HIV-induced encephalopathy and dementia |
| intermediates | 3. Ischemia-reperfusion injury (myocardial |
| | infarction, cerebrovascular accident/stroke) |
| Production of ONOO⁻ | 1. HIV infection/AIDS |
| (peroxynitrite) and reactive oxygen | 2. CMV infection |
| intermediates, resulting in reduced α1- | 3. Herpes simplex 1 and 2 infections |
| antitrypsin activity | 4. Influenza infection |
| | 5. Apoptosis-associated diseases |
| Direct toxicity | Neurotoxicity |
| Epithelial Damage | 1. Cystic fibrosis |
| | 2. Interstitial pulmonary fibrosis |
| Inflammation | 1. Asthma |
| | 2. Pulmonary embolism |

### Therapeutic Methods

Some aspects of the invention provide methods for inhibiting NO production for therapeutic benefits. Nitric oxide activity can be associated with inflammation, septic shock, adverse consequences of ischemia and reperfusion injury, hypotension, and cell death, to mention a few indications.

Inflammation involves cell-mediated immune response, with release of toxic molecules including NO. Of particular importance in the inflammatory response are macrophagic cells and endothelium, and some embodiments of the invention are directed to inhibiting NO production by these cells. Cell mediated immune response can be beneficial, e.g., for destroying infectious microorganisms such as bacteria and parasites, and for eliminating cancerous or virally infected cells. However, inflammation can become chronic, autoimmune, and detrimental. Therefore, other aspects of the invention provide methods and compositions for treating inflammation, for example, lung inflammation including, but not limited to, asthma; liver inflammation; acne; inflammatory bowel disease; arthritis; and the like. NO inhibitory activity of the molecules of the invention can be administered either as a primary therapy or in conjunction with other anti-inflammatory therapies, including, but not limited to, steroid treatment, immune-cell targeted antibody therapy, and the like.

Septic shock results from the host response to systemic bacterial infection, particularly to bacterial endotoxins, such as Gram negative lipopolysaccharides. Nitric oxide overproduction contributes to septic shock. Any reduction in NO production has an ameliorating effect on the symptoms of septic shock. Accordingly, some aspects of the invention provide methods for treating septic shock by administering α₁-antitrypsin, a derivative thereof, or a combination thereof. Some embodiments within these aspects of the invention include administering α₁-antitrypsin, a derivative thereof, or a combination thereof in conjunction with other therapies, e.g., antibodies to lipopolysaccharide, antibodies to tumor necrosis factor or interleukin-1, interleukin-1 receptor antagonist, or soluble TNF or IL-1 receptors. Macrophages and endothelium are particular cellular targets for inhibition of NO activity. To date, septic shock in humans has proved to be highly refractory to therapy. Therefore, it is a particular advantage of the invention to provide a therapy or co-therapy for septic shock.

NO has been associated with the adverse effects of ischemic events. Ischemia, or reduced blood perfusion of tissues, results in hypoxia and is a particularly serious problem when it occurs in the heart, e.g., as a consequence of myocardial infarct or after balloon angioplasty; in the brain, e.g., as a consequence of stroke; in the lungs; and in the kidneys. Therefore, administration of a dosage of the invention would greatly benefit a subject suspected of suffering from ischemia or reperfusion injury. Typically, methods of the invention provide administering a therapeutically effective amount of α₁-antitrypsin, a derivative thereof, or a combination thereof prior to or concomitant with any drugs designed to release the blockage causing the ischemic condition. In one specific embodiment, α₁-antitrypsin, a derivative thereof, or a combination thereof is administered prior to, or with, tissue plasminogen activator (tPA), streptokinase, and the like for treating myocardial infarct. The combination of α₁-antitrypsin, a derivative thereof, or a combination thereof, with tPA, streptokinase, and the like, can reduce inflammation and NO production and apoptosis associated with the infarct because NO and free radical production occur during ischemia/reperfusion. α₁-Antitrypsin, a derivative thereof, or a combination thereof, NOS inhibitor and/or other agents are advantageously administered within about the first four hours of ischemia, typically within the first hour after ischemia, and often concurrent with the ischemic event. These same inhibitors can also be administered prior to an anticipated ischemic event. Ischemic events can be anticipated in some patients in groups at risk. Patients under going angioplasty are in such a category, and patients undergoing many other types of surgery have an elevated risk. Also, patients who are at risk because of clotting disorders, arteriosclerosis, or a history of transient ischemic attacks (TIAs) are suitable candidates for preventative treatment. Patients in a high risk category for ischemia can be treated chronically. Endogenous α1-antitrypsin can be inactivated, e.g., by NO and free radicals, during reperfusion. This loss of α1-antitrypsin activity exacerbates NO production, inflammation, and apoptosis. Therefore, administration of exogenous α1-antitrypsin, an oxidation-resistant mutant α1-antitrypsin, or an oxidation-resistant synthetic analog are especially beneficial.

Hypotension, or low blood pressure, can cause problems with circulation. Hypotension and shock can result from sepsis, severe blood loss, serious organ injury, severe trauma and chemotherapy, particularly cytokine-based chemotherapy. Thus, the present invention provides for treatment of severe hypotension. In a specific embodiment, priapism (impotence) associated with hypotension can be treated. In another specific embodiment, hypotensive shock that may result from administration of IL-2 or TNF to treat cancer can be ameliorated. In ischemic injury, NO induces neurotoxicity. An embodiment of this invention reduces neurotoxicity by administration of inhibitors of NOSs and/or by administration of NO inhibitors, e.g., α₁-antitrypsin, a derivative thereof, or a combination thereof.

NO is an active neurotransmitter. Excessive production or activity of NO may result in neurological diseases, particularly those affecting the brain. Therefore, administration of a dosage of the invention composition, i.e., (α₁-antitrypsin, a derivative thereof, or a combination thereof), is beneficial for the treatment of neurological diseases or disorders. In some aspects of the invention, a derivative of α₁-antitrypsin is an analog of α₁-antitrypsin that can cross the blood brain barrier, which allows intravenous or oral administration. Many strategies are available for crossing the blood brain barrier including, but not limited to, increasing the hydrophobic nature of a molecule; introducing the molecule as a conjugate to a carrier, such as transferrin, targeted to a receptor in the blood brain barrier; and the like. In some embodiments, compositions of the invention is administered intracranially or, more directly, intraventricularly.

In a further embodiment, the methods and compositions of the invention are useful in the therapeutic treatment of diseases or disorders of the kidney. Glomerulonephritis is characterized by enhanced production of NO, which is believed to contribute to tissue injury. During inflammation, reperfusion, or other stress related processes, kidney cells are exposed to an array of factors and mediators that can stimulate excessive NO production. Excessive NO production results in increases in reactive intermediates, which can damage kidney tissues. Enhanced NO production is also a serious consequence of uremia. Thus, other aspects of the invention provide methods for ameliorating or alleviating many diseases of the kidney.

Ischemia-induced lung injury (shock lung), also known as acute respiratory distress syndrome, is a candidate for therapeutic intervention using α₁-antitrypsin, a derivative thereof, or a combination thereof, especially α₁-antitrypsin derivatives that are resistant to inactivation by reactive oxygen intermediates.

Certain metastatic diseases can also be treated by administration of α₁-antitrypsin, a derivative thereof, or a combination thereof. For example, inhibition of NO activity, which can result in reduced blood flow, aid in a treatment of solid tumors that involves or is enhanced by hypoxia.

Methods and compositions of the invention are also useful for the treatment of altitude sickness. Altitude sickness, among other contributing factors, is a result of reduced oxygen tension and consequential hypoxia of certain tissues, particularly the lungs and brain. Other aspects of the invention provide methods for alleviating the symptoms of altitude sickness by administering α₁-antitrypsin, a derivative thereof, or a combination thereof.

Still other aspects of the invention provide methods for treating a clinical condition associated with NO by administering a substance that increases α₁-antitrypsin expression rather than by directly administering α₁-antitrypsin.

In a yet other aspects of the invention, diseases are prevented by the timely administration of α₁-antitrypsin, a derivative thereof, or a combination thereof as a prophylactic, prior to onset of symptoms, or signs, or prior to onset of severe symptoms or signs. Thus, a patient at risk for a particular disease caused in part by excessive NO levels or excessive NOS expression, can be treated with α₁-antitrypsin, a derivative thereof, or a combination thereof as a precautionary measure.

The effective dose of the agent of the invention, and the appropriate treatment regiment, can vary with the indication and patient condition, and the nature of the molecule itself, e.g., its in vivo half life and level of activity. These parameters are readily addressed by one of ordinary skill in the art and can be determined by routine experimentation.

The physician will determine the dosage of the present therapeutic agents which will be most suitable for prophylaxis or treatment and it will vary with the form of administration and the particular compound chosen, and also, it will vary with the particular patient under treatment. The therapeutic dosage can generally be from about 0.1 to about 1000 mg/day, and typically from about 10 to about 100 mg/day, or from about 0.1 to about 100 mg/Kg of body weight total and often from about 0.1 to about 20 mg/Kg of body weight total and can be administered in several different dosage units. Higher dosages, on the order of about 2× to about 4×, may be required for oral administration. In some instances, typical doses for administration can be anywhere in a range between about 0.01 mg and about 20 mg per mL of biologic fluid of treated patient. The therapeutically effective amount of α₁-antitrypsin, a derivative thereof, or a combination thereof can also be measured in molar concentrations and can range between about 1 nM to about 2 mM.

In some instances, a mechanical device is used to reestablish blood flow in conjunction with administration of α₁-antitrypsin, a derivative thereof, or a combination thereof. The mechanical device can be, for example, a stent, or involve, for example, percutaneous transluminal coronary angioplasty (PTCA) or angioplasty.

### Modes of Administration

Modes of administering a composition comprising α₁-antitrypsin, a derivative thereof, or a combination thereof are exemplified below. However, the compositions of the invention can be delivered by any of a variety of routes including: by injection (e.g., subcutaneous, intramuscular, intravenous, intraarterial, intraperitoneal), by continuous intravenous infusion, transdermally, orally (e.g., tablet, pill, liquid medicine), by implanted osmotic pumps (e.g., Alza Corp.), by suppository or aerosol spray (metered dose inhaler or dry powder inhaler).

Derivatives of α₁-antitrypsin as well as α₁-antitrypsin itself can be prepared by any suitable synthesis method known to one skilled in the art including using recombinant DNA strategy as well as by chemical means using a solid phase synthesis such as those described by Merrifield, J. Am. Chem. Soc., 1963, 85, 2149.

Those skilled in the art of biochemical synthesis will recognize that for commercial scale quantities of α₁-antitrypsin or a derivative thereof, such peptides are generally prepared using recombinant DNA techniques, synthetic techniques, or chemical derivatization of biologically or chemically synthesized peptides.

The compounds of the present invention are used as therapeutic agents in the treatment of a physiological (especially pathological) condition caused in whole or part, by NO activity. The peptides may be administered as free peptides or pharmaceutically acceptable salts thereof. The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes of the peptides which do not significantly or adversely affect the therapeutic properties (e.g. efficacy, toxicity, etc.) of the peptides. The peptides should be administered to individuals as a pharmaceutical composition, which, in most cases, comprise the peptide and/or pharmaceutical salts thereof with a pharmaceutically acceptable carrier. The term “pharmaceutically acceptable carrier” refers to those solid and liquid carriers that do not significantly or adversely affect the therapeutic properties of the peptides.

The pharmaceutical compositions containing peptides of the present invention can be administered to individuals, particularly humans, using any of the methods known to one skilled in the art including, but not limited to, intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration can be accomplished by application of a cream, rinse, gel, etc. capable of allowing α₁-antitrypsin, a derivative thereof, or a combination thereof to penetrate the skin and enter the blood stream. Parenteralroutes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, or endotracheal tube. In addition, osmotic pumps can be used for administration. The necessary dosage will typically vary with the particular condition being treated, method of administration and rate of clearance of the molecule from the body.

Although the compounds described herein and/or their derivatives can be administered as the pure chemicals, typically the active ingredient as administered as a pharmaceutical composition. Thus, some embodiments of the invention provide methods for using a pharmaceutical composition comprising one or more compounds and/or a pharmaceutically acceptable salt thereof, together with one or more pharmaceutically acceptable carriers therefor and, optionally, other therapeutic and/or prophylactic ingredients. The carrier(s), when used, are compatible with the other ingredients of the composition and not deleterious to the recipient thereof.

Pharmaceutical compositions include those suitable for oral or parenteral (including intramuscular, subcutaneous and intravenous) administration. The compositions can, where appropriate, be conveniently presented in discrete unit dosage forms and can be prepared by any of the methods well known in the art. Such methods include the step of bringing into association the active compound with liquid carriers, solid matrices, semi-solid carriers, finely divided solid carriers or combinations thereof, and then, if necessary, shaping the product into the desired delivery system.

Pharmaceutical compositions suitable for oral administration can be presented as discrete unit dosage forms such as hard or soft gelatin capsules, cachets or tablets, each containing a predetermined amount of the active ingredient; as a powder or as granules; as a solution, a suspension or as an emulsion. The active ingredient can also be presented as a bolus, electuary or paste. Tablets and capsules for oral administration can also contain conventional excipients such as binding agents, fillers, lubricants, disintegrants, or wetting agents. The tablets can also be coated according to methods well known in the art, e.g., with enteric coatings.

Oral liquid preparations can be in the form of, for example, aqueous or oily suspension, solutions, emulsions, syrups or elixirs, or can be presented as a dry product for constitution with water or another suitable vehicle before use. Such liquid preparations may contain conventional additives such as suspending agents, emulsifying agents, non-aqueous vehicles (which can include edible oils), or preservative.

The compounds can also be formulated for parenteral administration (e.g., by injection, for example, bolus injection or continuous infusion) and can be presented in unit dose form in ampoules, pre-filled syringes, small bolus infusion containers or in multi-dose containers with an added preservative. The compositions can take such forms as suspensions, solutions, or emulsions in oily or aqueous vehicles, and can also contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For topical administration to the epidermis, the compounds can be formulated as ointments, creams or lotions, or as the active ingredient of a transdermal patch. Suitable transdermal delivery systems are disclosed, for example, in U.S. Pat. Nos. 4,788,603; 4,931,279; and 4,713,224. Ointments and creams can, for example, be formulated with an aqueous or oily base with the addition of suitable thickening and/or gelling agents. Lotions can be formulated with an aqueous or oily base and typically also contain one or more of the following: emulsifying agents, stabilizing agents, dispersing agents, suspending agents, thickening agents, and coloring agents. The active ingredient can also be delivered via iontophoresis, e.g., as disclosed in U.S. Pat. Nos. 4,140,122; 4,383,529; and 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; muco adherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the patient and will be selected, ultimately, at the discretion of the attendant physician.

A pharmaceutical composition of the invention contains an appropriate pharmaceutically acceptable carrier as defined supra. These compositions can take the form of solutions, suspensions, tablets, pills, capsules, powders, sustained-release formulations and the like. Suitable pharmaceutical carriers are described in Remington's Pharmaceutical Sciences 1990, pp. 1519-1675, Gennaro, A. R., ed., Mack Publishing Company, Easton, Pa. α₁-Antitrypsin, a derivative thereof, or a combination thereof can be administered in liposomes or polymers (see, Langer, R., Nature, 1998, 392, 5). Such compositions contain an effective therapeutic amount of the active compound together with a suitable amount of carrier so as to provide the form for proper administration to the subject.

In general, the compound is conveniently administered in unit dosage form; for example, containing 5 to 2000 mg, conveniently 10 to 1000 mg, most conveniently, 50 to 500 mg of active ingredient per unit dosage form.

Desirable blood levels can be maintained by continuous infusion to provide about 0.01-60.0 mg/kg/hr or by intermittent infusions containing about 0.4-20 mg/kg of the active ingredient(s). Buffers, preservatives, antioxidants and the like can be incorporated as required.

The desired dose can conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

The following specific examples are provided to better assist the reader in the various aspects of practicing the present invention. As these specific examples are merely illustrative, nothing in the following descriptions should be construed as limiting the invention in any way. Such limitations are, of course, defined solely by the accompanying claims.

Effect of α₁-Antitrypsin on Nitric oxide (NO) Production

RAW 264.7 macrophages were selected for measuring the effect of α₁-antitrypsin on NO release. RAW 264.7 cell monolayers were pretreated for 1 hour with α₁-antitrypsin (0.1-3 mg/mL), followed by costimulation by interferon-γ (10 U/mL), and LPS (1 ng/mL) for 18 hours. Aliquots (100 μL) of supernatant were combined with equal volumes of Greiss reagent and incubated at room temperature for 10 minutes. The calorimetric determination of nitrite concentration was measured by absorbance at 550 nm and quantified with a standard curve. The combination of LPS and interferon-γ was a potent stimulus for NO release in RAW 264.7 macrophages. The effect of α₁-antitrypsin on NO expression was measured.

### Combined Effect of α₁-Antitrypsin and an Antioxidant on Nitric Oxide (NO) Production

RAW 264.7 cell monolayers were pretreated for 1 hour with seven concentrations of α₁-antitrypsin (0.003, 0.01, 0.03, 0.1, 0.3, 1, and 3 mg/mL) in the absence or the presence of β-carotene (1 mg/mL), followed by costimulation by interferon-γ (10 U/mL), and LPS (1 ng/mL) for 18 hours. Aliquots (100 μL) of supernatant were combined with equal volumes of Greiss reagent and incubated at room temperature for 10 minutes. The colorimetric determination of nitrite concentration was measured by absorbance at 550 nm and quantified with a standard curve. The effect of α₁-antitrypsin in combination with β-carotene on NO release was compared to the effect of each agent individually.

### Combined Effect of α₁-Antitrypsin and a Free Radical Scavenger on NO Production

RAW 264.7 cell monolayers were pretreated for 1 hour with seven concentrations of α₁-antitrypsin (0.003, 0.01, 0.03, 0.1, 0.3, 1, and 3 mg/mL) in the absence or the to presence of 2,6,8-trihydroxypurine (0.1 mg/mL), followed by costimulation by interferon-γ (10 U/mL), and LPS (1 ng/mL) for 18 hours. Aliquots (100 μL) of supernatant were combined with equal volumes of Greiss reagent and incubated at room temperature for 10 minutes. The colorimetric determination of nitrite concentration was measured by absorbance at 550 nm and quantified with a standard curve. The combination of LPS and interferon-γ produced a powerful stimulus for NO release in RAW264.5 macrophages.

The effect of α₁-antitrypsin in combination with 2,6,8-trihydroxypurine was compared to the effect of each agent individually.

### Inhibition of INOS Induction.

RAW 264.7 macrophage monolayers were treated for 1 hour with α₁-antitrypsin (3 mg/mL), followed by costimulation by interferon-γ (10 U/ml), and LPS (1 ng/mL) for 18 hours. The cells were lysed by exposure to lysis solution (50 mm Tris-HCl, pH 8.0, 137 mm NaCl, 10% (v/v) glycerol, 1% (v/v), Nonidet P-40, 1 mM NaF, 10 μg/mL leupeptin, 10 mg/mL aprotinin, 2 mM sodium vanadate, and 1 mM phenylmethylsulfonyl fluoride). Samples containing equivalent amounts of total protein were subjected to SDS-polyacrylamide gel electrophoresis. Western blots of the gels were prepared, non-specific sites blocked by incubation overnight with 5% non-fat dry milk, and iNOS were detected by incubation with iNOS anti-serum (Alexis Corporation, 1:1000 in 5% (w/v) bovine serum albumin in a solution of 20 mm Tris-HCl, pH 7.6, 137 mM MgCl, and 0.005% (v/v) Tween 20). Using horseradish peroxidase-conjugated second antibody, the antibody bound to iNOS was detected by enhanced chemiluminescence. The effect of the combination of interferon-γ, and LPS on induction of iNOS in the cell extract and the effect of pretreatment with α₁-antitrypsin were measured.

### α₁-Antitrypsin in Experimental Allergic Encephalomyelitis.

Induction of Experimental Allergic Encephalomyelitis (EAE), a model for multiple sclerosis, in rats by adoptive transfer of myelin basic protein (MBP)-specific T cells or in SJL or SWXJ-14 mice by immunization with MBP or proteolytic protein from the myelin sheath (PLP 139-151), a peptide derived from MBP, resulted in variable disease. The clinical symptoms of EAE were scored as tabulated below.

TABLE 2
Severity Scores and Symptoms of Experimental Allergic
Encephalomyelitis Score Clinical Symptoms

1 piloerection, tail weakness
2 tail paralysis
3 hindlimb weakness/paralysis
4 hind and forelimb paralysis
5 moribund

The severity of clinical symptoms of EAE was determined in relation to NO production in the CNS. The site of major NO production is known to vary between different EAE models. The adoptive transfer of MBP-specific T cells in Lewis rats caused NO production which was largely limited to the spinal cord while immunization of SWXJ-14 mice with PLP 139-151 resulted in the elaboration of high levels of NO in both spinal cord and brain. Mice (n=3) were treated beginning on day 5 post-immunization with 2 mg/mouse α₁-antitrypsin once daily i.p. and were continued until day 16 after the immunization. Mean severity scores were graded as detailed in Table 2.

### α₁-Antitrypsin Effect on N-CNOS and E-CNOS

A soluble cytosolic fraction of the rat cerebral cortex was used as a source of N-cNOS. An homogenate of bovine pulmonary arterial endothelium (BPAE) cells was used as a source of E-cNOS. The following NOS inhibitors were used as control compounds: L-NNA; N^{G}-nitro-L-arginine methyl ester (L-NAME); N^{G}-amino-L-arginine (L-AA); N^{G}-iminoethyl-ornithine (L-NIO); N^{G}-monomethyl-L-arginine (L-NMMA); N^{G}-allyl-L-arginine (L-ALA); and 7-nitroindazole (7-NI); aminoguanidine (AG).

The N-cNOS crude enzyme was prepared by the following procedure. The whole brains of normal untreated male Sprague-Dawley (SD) rats weighing 300-400 g were homogenized for 3 min in 5 volumes of cold solution: 50 mM Tris-HCl containing 1 mM DTT (pH 7.4), followed by centrifugation at 1,000×g for 10 min. The supernatant was further centrifuged at 100,000×g for 60 min and a soluble cytosolic fraction of the finally obtained supernatant was used as the source of N-cNOS.

The crude enzyme sample of E-cNOS was prepared by the following procedure. BPAE cells were cultured in MEM medium containing 20% of fetal bovine serum. When the cells were confluent, the cells were detached from the flask using a solution of 0.25% trypsin containing 1 mM EDTA in 0.1 M phosphate-buffered saline (PBS; pH 7.4) and centrifuged at 1,000 rpm for 5 min. The supernatant was discarded and upon addition of a suitable amount of PBS, centrifugation was performed at 1,000 rpm for 5 min to wash the cells. The same procedure was repeated using 50 mM Tris-HCl containing 1 mM DTT (pH 7.4) to wash the cells. To the precipitating cells, there was added 50 mM Tris-HCl containing 1 mM DTT (pH 7.4) and the mixture was homogenized for 3 min to yield the crude enzyme sample of E-cNOS. An inhibitor of serine proteases, e.g., (benzyloxycarbonyl)-L-valyl-N-[1-(3-(-5-(3-trifluoromethylbenzyl)-1,2,4-oxadiazolyl)carbonyl)-2-(S)-methylpropyl]-L-prolinamide; (5 mg/mL) or one of the control compounds, was added to the reaction solution, consisting of 100 nM L-[³H] arginine, N-cNOS or E-cNOS as crude enzyme sample (6-20 μg/mL protein), 1.25 mM CaCl₂, 1 mM EDTA, 10 μg/mL calmodulin, 1 mM NADPH, 100 μM tetrahydrobiopterin, 10 μM FAD, 10 μM FMN and 50 mM Tris-HCl (pH 7.4).

The reaction was started by adding the L-[³H] arginine to the reaction solution and the mixture was incubated at 37° C. for 10 min. Incubation was terminated by addition of 2 mL of 50 mMTris-HCl (pH 5.5) containing 1 mM EDTA. The reaction solution was quenched by placing the mixture on ice. The reaction solution was passed through a cation-exchange resin column (Dowex AG50WX-8, Na⁺ form, 3.2 mL) and the reaction product L-[³H] citrulline was separated from the unreacted residual substrate L-[³H] arginine. The eluant was combined with another eluant resulting from the passage of distilled water (3 mL) through the column and put into a mini vial for recovery of L-[³H] citrulline. Thereafter, 5 mL of a scintillation fluid was added and the contained radioactivity was measured with a liquid scintillation counter to determine the amount of L-[³H] citrulline. The protein concentration of each crude enzyme sample was determined with a micro-assay kit of BioRad Co.

### An In Vitro Model for Septic Shock

The effects of the agents α₁-antitrypsin, (benzyloxycarbonyl)-L-valyl-N-[1-(3-(5-(3-trifluoromethylbenzyl)-1,2,4-oxadiazolyl)carbonyl)-2-(S)-methylpropy-l]-L-prolinamide; (benzyloxycarbonyl)-L-valyl-N-[1-(3-(5-(2-phenylethyl)-1-,2,4-oxadiazolyl)carbonyl)-2-(S)-methylpropyl]-L-prolinamide; and (benzyloxycarbonyl)-L-valyl-N-[1-(3-(5-(2-methoxybenzyl)-1,2,4-oxadiazoly-1)carbonyl)-2-(S)-methylpropyl]-L-prolinamide for protection of mouse L929 cells from cytotoxic effects of TNF are evaluated as follows. L929 cells (10⁵ cells/well) are treated with 300 ng/mL of human TNF with or without the agent (added one hour prior to TNF addition) at 0.03, 0.1, 0.3, 1.0, 3.0 and 10 mg agent/mL. One day later the cells are stained for viability using 2′,7′-bis(2-carboxyethyl)-5(6)′-carboxyfluorescein and fluorescence analyzed for viability using a Millipore fluorescence plate reader. The results are evaluated in terms of the dose response to the agent.

### Effect of Protease Inhibitor Agents on γ-IFN Stimulation of Monocyte Production of Cytokines

The effect of the agents α₁-antitrypsin, (benzyloxycarbonyl)-L-valyl-N-[1-(3-(-5-(3-trifluoromethylbenzyl)-1,2,4-oxadiazolyl)carbonyl)-2-(S)-methylpropyl-]-L-prolinamide; (benzyloxycarbonyl)-L-valyl-N-[1-(3-(5-(2-phenylethyl)-1-,2,4-oxadiazolyl)carbonyl)-2-(S)-methylpropyl]-L-prolinamide; (benzyloxycarbonyl)-L-valyl-N-[1-(3-(5-(2-methoxybenzyl)-1,2,4-oxadiazoly-l)carbonyl)-2-(S)-methylpropyl]-L-prolinamide; (3 mg/mL) on cytokine production by monocytes activated by γ-IFN (100 U/mL), or combinations of γ-IFN and LPS (1 μg/mL) is evaluated. HL-60 monocyte-like cells are aliquoted into microwell plates (10⁵ cells/well) and treated in the presence of saline, γ-IFN (100 U/mL), LPS (1 μg/mL), or combinations of γ-IFN and LPS for 24 hrs at 37° C. The conditioned media are collected and assayed for interleukin (IL)-1α, tumor necrosis factor (TNF)-α, and granulocyte-macrophage colony stimulating factor (GM-CSF) production by ELISA. The rank order of efficacy of the agents is determined for production of each cytokine.

### Protease Inhibitor Agent Effects in LPS-Induced Inflammation

LPS (250 μg. E. coli K-235, Sigma cat. no. L-2018) is administered to normal BALB/c mice (female, 12 weeks) at time zero. One group of mice (50 animals) is then treated at 30 minute intervals by i.p. injections of bovine serum albumin (BSA) (Sigma cat. no. 6793) dissolved in pyrogen-free, sterile, isotonic water (2.5 mg BSA per animal per injection, each injection containing 100 μL). The second group of mice (50 animals) is treated at 30 minutes intervals by i.p. injections of (benzyloxycarbonyl)-L-valyl-N-[1-(3-(5-(3-trifluoromethylbenzyl)-1,2,4-ox-adiazolyl)carbonyl)-2-(S)-methylpropyl]-L-prolinamide dissolved in pyrogen-free, sterile, isotonic water (0.2 mL per animal per injection, each injection 3 mg/mL). Glucose levels are determined on blood samples at time zero and after 3 hours, as a measure of response to LPS and to the agent.

### Effects of α₁-Antitrypsin and Other Compounds in a Model of Endotoxemia.

Swiss-Webster mice 4-6 weeks of age (20-25 g) are divided into 5 groups: endotoxic mice (endotoxin 60 mg/kg i.p. in acute treatment); two groups of endotoxic mice treated with 3 injections of 100 μL α₁-antitrypsin (5 minutes, 2 and 4 hours post administration of the endotoxin) at α₁-antitrypsin concentrations of 10 mg/mL and 1 mg/mL, respectively; and two groups of endotoxic mice treated with 3 injections of 100 μL (benzyloxycarbonyl)-L-valyl-N-[1-(3-(5-(3-trifluoromethylbenzyl)-1,2,4-oxadiazolyl)carbonyl)-2-(S)-methylpropyl]-L-prolinamide (5 minutes, 2 and 4 hours post administration of the endotoxin) at agent concentrations of 5 mg/mL and 1 mg/mL, respectively. The effect of the protease inhibitors on the survival rate, and on blood levels of malonyldialdehyde, glutathione, TNF-α, and IL-1α is measured.

### Effects of α₁-Antitrypsin and Other Agents in a Model of Septic Shock.

Peritonitis is induced in rats (Sprague-Dawley, male, 200-225 g each) in the following way. A one cm incision is made into the peritoneum to expose the cecum. A tight ligature is placed around the cecum with 4-0 suture distal to the insertion of the small bowel, forming an area of devitalized tissue while maintaining bowel continuity. A puncture wound is made with 16-gauge needle into the anti-mesenteric surface of the cecum and a small amount of fecal contents is expressed through the wound. The cecum is replaced into the peritoneal cavity, and the anterior peritoneal wall and skin are closed with surgical staples. Each animal is given a bolus of normal saline (15 mL/kg) for hydration and allowed to recover overnight. At 24 hours a schedule of treatment is initiated, with injections at 6 hr intervals. One group of animals is injected with 0.5 mL saline, another group is injected (i.p.) with 0.5 mL of α₁-antitrypsin (5 mg/mL); and a third group is injected (i.p.) with 0.5 mL of (benzyloxycarbonyl)-L-valyl-N-[1-(3-(5-(difluoromethyl-)-1,2,4-oxadiazolyl)carbonyl)-2-(S)-methylpropyl]-L-prolinamide (5 mg/mL on each day). The seven-day survival rate is measured.

### Modulation of Proteinase-Activated Receptors

The invention also relates to the effect of α₁-antitrypsin and α₁-antitrypsin-like agents on the activation of proteinase-activated receptors (PARs). Alpha₁-antitrypsin and α₁-antitrypsin-like agents block PAR activation and thereby reduce vasodilation mediated by NO, reduce extravasation of plasma proteins, decrease infiltration of immune cells, and block protease-stimulated mitosis. Thus the diseases described above can be treated with inhibitors of PAR, including, but not limited to, α₁-antitrypsin, α₁-antitrypsin-like agents, blocking antibodies, inhibitory kinases and kinase cDNA, inhibitory proteases, and hirudin. Inhibitory proteases can include any protease that cleaves the PAR at a site other than the activation site.

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of treating a subject suffering from a clinical condition associated with elevated synthesis of nitric oxide, said method comprising administering to the subject in need of such a treatment a composition comprising a therapeutically effective amount of α₁-antitrypsin, a derivative thereof, or a combination thereof.

2. The method of claim 1, wherein the clinical condition associated with elevated synthesis of nitric oxide comprises a clinical condition associated with γ-IFN-induced NO synthesis, LPS-induced NO synthesis, or a combination thereof.

3. The method of claim 1, wherein the clinical condition associated with elevated synthesis of nitric oxide comprises induced inflammation, COPD, asthma, burn injury, bacterial infection, fungal infection, parasitic infection, high altitude sickness, HAPE and HACE edema, endotoxemia, ischemia reperfusion injury, acute or chronic bronchitis, sinusitis, upper respiratory infections, acute or chronic cystitis, urethritis, acute or chronic dermatitis; acute or chronic conjunctivitis; acute or chronic serositis, uremic pericarditis, acute or chronic cholecystitis, acute or chronic vaginitis, drug reactions, insect bites, burns, sunburn, or a combination thereof.

4. The method of claim 1, wherein the clinical condition associated with elevated synthesis of nitric oxide comprises sepsis, septic shock, ARDS (shock lung), acute renal failure, shock liver, acute ischemic bowel injury, myocardial depression of sepsis, acute and chronic congestive heart failure, neurotoxicity, ischemic brain injury, HIV-induced encephalopathy and dementia, ischemia-reperfusion injury, cystic fibrosis, interstitial pulmonary fibrosis, asthma, pulmonary embolism.

5. The method of claim 4, wherein the ischemia-reperfusion injury is associated with heart, brain, lung, kidneys, liver, gastrointestinal tract, limbs, digits, or coagulation.

6. The method of claim 5, wherein the ischemia-reperfusion injury comprises myocardial infarction, cerebrovascular accident/stroke, angina/chest pain, atypical angina, unstable angina, coronary artery disease, atherosclerosis, ischemic cardiomyopathy, transient ischemic attack (TIA), intracerebral hemorrhage, acute respiratory distress syndrome (ARDS), shock lung, shock liver, pre-renal azotemia, ischemic nephropathy, glomerulonephritis, ischemic bowel, bowel infarction, limb ischemia, limb infarction, thromboangiitis obliterans, Raynaud phenomenon, Raynaud disease, or disseminated intravascular coagulopathy (DIC).

7. The method of claim 1, wherein the composition comprises a therapeutically effective amount of α₁-antitrypsin.

8. The method of claim 1, wherein the composition further comprises an inhibitor of NO synthesis selected from the group consisting of N^{G}-nitro-L-arginine methyl ester (L-NAME), N^{G}-nitro-L-arginine(L-NA), N^{G}-methyl-L-arginine (L-NMA), N,N′-dimethylarginine, N^{G}-monoethyl-L-arginine acetate, N^{G}-monomethyl-L-arginine acetate, N^{G}-monomethyl-D-arginine, N^{G}-monomethyl-L-homoarginine acetate, N N^{G}-nitro-D-arginine, N^{G}-nitro-D-arginine methyl ester hydrochloride, N^{G}-nitro-L-arginine, and L-N⁶-(1-iminoethyl)lysine, guanidine, guanidine derivatives, S-alkylisothioureas, amidines, imidazoles, indazoles, and mercaptoalkylguanidines, and salts thereof.

9. The method of claim 1, wherein the composition further comprises a vasoconstrictor.

10. A method for treating a subject for an ischemia-reperfusion injury associated with heart, brain, lung, kidneys, liver, gastrointestinal tract, limbs, or coagulation, said method comprising administering to the subject in need of such a treatment a composition comprising a therapeutically effective amount of α₁-antitrypsin, a derivative thereof, or a combination thereof.

11. The method of claim 10, wherein the ischemia-reperfusion injury comprises myocardial infarction, cerebrovascular accident/stroke, angina/chest pain, atypical angina, unstable angina, coronary artery disease, atherosclerosis, ischemic cardiomyopathy, transient ischemic attack (TIA), intracerebral hemorrhage, acute respiratory distress syndrome (ARDS), shock lung, shock liver, pre-renal azotemia, ischemic nephropathy, glomerulonephritis, ischemic bowel, bowel infarction, limb ischemia, limb infarction, thromboangiitis obliterans, Raynaud phenomenon, Raynaud disease, or disseminated intravascular coagulopathy (DIC).

12. The method of claim 10, wherein the composition comprises a therapeutically effective amount of α₁-antitrypsin.

13. The method of claim 10, wherein the composition further comprises an inhibitor of NO synthesis selected from the group consisting of N^{G}-nitro-L-arginine methyl ester (L-NAME), N^{G}-nitro-L-arginine(L-NA), N^{G}-methyl-L-arginine (L-NMA), N,N′-dimethylarginine, N^{G}-monoethyl-L-arginine acetate, N^{G}-monomethyl-L-arginine acetate, N^{G}-monomethyl-D-arginine, N^{G}-monomethyl-L-homoarginine acetate, N N^{G}-nitro-D-arginine, N^{G}-nitro-D-arginine methyl ester hydrochloride, N^{G}-nitro-L-arginine, and L-N^{G}-(1-iminoethyl)lysine, guanidine, guanidine derivatives, S-alkylisothioureas, amidines, imidazoles, indazoles, and mercaptoalkylguanidines, and salts thereof.

14. The method of claim 10, wherein the composition further comprises a vasoconstrictor.

15. A method for treating a clinical condition associated with elevated synthesis of nitric oxide comprising induced inflammation, COPD, asthma, burn injury, bacterial infection, fungal infection, parasitic infection, high altitude sickness, HAPE and HACE edema, endotoxemia, acute or chronic bronchitis, sinusitis, upper respiratory infections, acute or chronic cystitis, urethritis, acute or chronic dermatitis; acute or chronic conjunctivitis; acute or chronic serositis, uremic pericarditis, acute or chronic cholecystitis, acute or chronic vaginitis, drug reactions, insect bites, burns, sunburn, sepsis, septic shock, ARDS (shock lung), acute renal failure, shock liver, acute ischemic bowel injury, myocardial depression of sepsis, acute and chronic congestive heart failure, neurotoxicity, ischemic brain injury, HIV-induced encephalopathy and dementia, cystic fibrosis, interstitial pulmonary fibrosis, asthma, pulmonary embolism, or a combination thereof, said method comprising administering to the subject in need of such a treatment a composition comprising a therapeutically effective amount of α₁-antitrypsin, a derivative thereof, or a combination thereof.

16. The method of claim 15, wherein the composition comprises a therapeutically effective amount of α₁-antitrypsin.
